(19) United States
(12) Patent Application Publication
(10) Pub. No.: US 2025/0201372 A1
(43) Pub. Date: Jun. 19, 2025

(54) SYSTEM AND METHOD PROVIDING GUIDANCE REGARDING CARDIOVASCULAR DISEASE PREVENTION ACCORDING TO MEDICAL GUIDELINES

(71) Applicants: Aditi Vaishnav, New York, NY (US); Sudhir Vaishnav, New York, NY (US)

(72) Inventors: Aditi Vaishnav, New York, NY (US); Sudhir Vaishnav, New York, NY (US)

(21) Appl. No.: 18/833,893

(22) PCT Filed: Dec. 23, 2022
(86) PCT No.: PCT/IB2022/062725

§ 371 Date: Jul. 27, 2024

(30) Foreign Application Priority Data
Jan. 27, 2022 (IN) 202221004420

(51) Int. Cl. G16H 20/10; G16H 10/60; G16H 70/20

(52) U.S. Cl. CPC G16H 20/10; G16H 10/60; G16H 70/20

(57) ABSTRACT

The present disclosure relates to a system (102) for providing guidance for cardiovascular disease prevention, the system comprising a processor (202) operatively coupled to a memory (204), the memory storing instructions executable by the processor (202) to receive a set of attributes from a user (108) associated with a computing device (106), analyse the received set of attributes to stratify the user (108) into one or more risk categories, wherein based on the stratification of the user into one or more risk categories, the processor is configured to assess medication needs for the user to help achieve lipid control targets recommended by medical guidelines.

### TECHNICAL FIELD

The present disclosure relates, in general, to diagnosis and treatment of cardiovascular conditions, and more specifically, relates to a system and method for providing guidance regarding cardiovascular disease prevention according to evidence-based medical guidelines.

### BACKGROUND

Despite significant advances in therapy, cardiovascular disease remains the most common cause of morbidity and mortality in the world. Heart disease specifically ischaemic heart disease i.e., cardiovascular disease has been the leading cause of death globally for the past 20 years, and the numbers continue to rise. However, most people with elevated risk and early-stage disease do not receive timely screening and timely prevention, even though there is a group of medications that have proven to reduce cardiovascular disease risk.

Even amongst those who begin prevention measures, the measures are usually not adequate, resulting in uncontrolled risk factors. The reasons for uncontrolled risk factors include:
People are not aware of the fact that heart disease begins developing 10-20 years before any symptoms develop. Therefore, they do not consult cardiologists when prevention is likely to be most effective and helpful.
The primary care physicians and non-cardiac doctors who see patients at an earlier stage frequently do not begin prevention because the process of cardiovascular disease screening and prevention depends on the individual doctor's knowledge of the subject, which can vary significantly.
Additionally, even if people start consulting cardiologists at an early stage, there would be a huge burden on cardiologists considering the extremely large number of patients.

Prior art in the field is focused on methods of risk assessment and risk monitoring. However, there is no system or provide guidance to individuals to help them reduce their risk on the basis of evidence-based medical guidelines.

There is a therefore a need in the art to address the above-mentioned limitations and other disadvantages by enabling a system for providing guidance regarding cardiovascular disease prevention based on national and international medical guidelines.

### Objects of the Present Disclosure

A general objective of the present disclosure relates, in general, to diagnosis and treatment of cardiovascular conditions, and more specifically, relates to a system and method for providing guidance regarding cardiovascular disease prevention according to evidence-based medical guidelines.

Another objective of the present disclosure is to provide a system that enables the user with elevated risk and early-stage disease to receive timely screening and timely prevention of cardiovascular disease.

Another objective of the present disclosure is to provide a system that removes the knowledge variability and equips all doctors to provide gold-standard cardiovascular disease prevention advice to all patients.

Another objective of the present disclosure is to provide a system that can ensure that all patients receive cardiologist-level of prevention even if they are being seen by primary care physicians or other non-physician providers (such as, Nurse Practitioners or Physician Assistants).

Another objective of the present disclosure is to provide a system that reduces the enormous burden that falls on the limited number of cardiologists.

Yet another objective of the present disclosure is to provide a system that serves as an educational tool for non-cardiac healthcare providers and common people to learn more about heart disease risk, risk factors, means to reduce risk and control risk factors.

### BRIEF DESCRIPTION OF THE DRAWINGS

In the figures, similar components and/or features may have the same reference label. Further, various components of the same type may be distinguished by following the reference label with a second label that distinguishes among the similar components. If only the first reference label is used in the specification, the description is applicable to any one of the similar components having the same first reference label irrespective of the second reference label.

FIG. 1 illustrates a network implementation 100 of a system which facilitates to provide guidance regarding cardiovascular disease prevention, in accordance with an embodiment of the present disclosure.

FIG. 2 illustrates exemplary functional components 200 of the proposed system in accordance with an embodiment of the present disclosure.

FIG. 3A illustrates a framework 300 for stratification of user into risk categories, in accordance with an embodiment of the present disclosure.

FIG. 3B illustrates a framework 302 for providing guidance regarding the appropriate type/intensity of statin and the recommended dose, in accordance with an embodiment of the present disclosure.

FIG. 3C illustrates a framework 304 for managing elevated triglycerides, in accordance with an embodiment of the present disclosure.

FIG. 4 illustrates an exemplary computer system 400 to implement the proposed system in accordance with embodiments of the present disclosure.

FIG. 5 illustrates a flow chart of method for facilitating guidance regarding cardiovascular disease prevention, in accordance with an embodiment of the present disclosure.

### SUMMARY

The present disclosure relates, in general, to diagnosis and treatment of cardiovascular conditions, and more specifically, relates to a system and method for providing guidance regarding cardiovascular disease prevention according to evidence-based medical guidelines.

In an aspect, the present disclosure provides a system for providing guidance of cardiovascular disease prevention, the system comprising a processor operatively coupled to a memory, the memory storing instructions executable by the processor to receive a set of attributes from a user associated with a computing device, the set of attributes pertaining to medical history information, blood test information, imaging test information, current medication and any combination thereof, analyse the received set of attributes to stratify the user into one or more risk categories, wherein based on the stratification of the user into one or more risk categories, the processor is configured to assess medication needs for the user to help achieve lipid control targets recommended by medical guidelines.

According to an embodiment, the risk percent of the cardiovascular disease is calculated using the appropriate risk scoring system available.

According to an embodiment, the processor configured to provide guidance to the user regarding the appropriate intensity of medications and the recommended dose.

According to an embodiment, the processor recommends changes to the medication according to targets for lipid control set by evidence-based national and international medical guidelines

According to an embodiment, the processor monitors adverse effects of the medications to ensure safety, wherein when the adverse effects are detected, appropriate action is recommended.

According to an embodiment, the processor assesses risk factors for elevated triglycerides and appropriate lifestyle and medications are recommended.

According to an embodiment, the processor configured to provide recommendation to the user to consult an expert specialist, whenever the clinical scenario depends on the clinical judgement of the specialist.

According to an embodiment, the medications is statin and any combination thereof.

In an aspect, the present disclosure provides a method for providing guidance of cardiovascular disease prevention, the method comprising receiving, at a processor, a set of attributes from a user associated with a computing device, the set of attributes pertaining to medical history information, blood test information, imaging test information, current medication and any combination thereof, analysing, at the processor, the received set of attributes to stratifying, at the processor, the user into one or more risk categories, wherein based on the stratification of the user into one or more risk categories, the processor is configured to assess medication needs for the user to help achieve lipid control targets recommended by medical guidelines.

Various objects, features, aspects, and advantages of the inventive subject matter will become more apparent from the following detailed description of preferred embodiments, along with the accompanying drawing figures in which like numerals represent like components.

### DETAILED DESCRIPTION

In the following description, numerous specific details are set forth in order to provide a thorough understanding of embodiments of the present invention. It will be apparent to one skilled in the art that embodiments of the present invention may be practiced without some of these specific details.

If the specification states a component or feature “may”, “can”, “could”, or “might” be included or have a characteristic, that particular component or feature is not required to be included or have the characteristic.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The present disclosure relates, in general, to diagnosis and treatment of cardiovascular conditions, and more specifically, relates to a system and method for providing guidance regarding cardiovascular disease prevention. The present disclosure provides system and method for facilitating guidance regarding cardiovascular disease prevention, based on national and international medical guidelines. The system and method of the present disclosure enable to overcome the limitation of the prior art by utilizing medical history, blood test and imaging test results of the user to provide preventive guidance/recommendations that are optimized according to each individual's risk level and medical condition. The system and method of the present disclosure removes the knowledge variability and equips all doctors to provide gold-standard cardiovascular disease prevention advice to all patients. It does this by replicating the diagnostic and therapeutic thought-process of an expert cardiologist in a digital format to the maximum extent possible while still ensuring patient safety.

The system and method of the present disclosure enable to overcome the limitation of the prior art by ensuring that all patients who require cardiovascular disease prevention receive cardiologist-level of prevention even if they are being seen by primary care physicians or other non-physician providers (such as, Nurse Practitioners or Physician Assistants). The system and method provide recommendations to the user to help achieve the lipid control targets recommended by medical guidelines thereby reducing the enormous burden that falls on the limited number of cardiologists. The term “lipid profile” as used herein relates to blood tests and other suitable test to determine abnormalities in lipids, such as cholesterol, triglycerides and the likes.

Additionally, it can serve as an educational tool for non-cardiac doctors and common people to learn more about heart disease risk, risk factors, reduce risk and control risk factors. The description of terms and features related to the present disclosure shall be clear from the embodiments that are illustrated and described; however, the invention is not limited to these embodiments only. Numerous modifications, changes, variations, substitutions, and equivalents of the embodiments are possible within the scope of the present disclosure. Additionally, the invention can include other embodiments that are within the scope of the claims but are not described in detail with respect to the following description

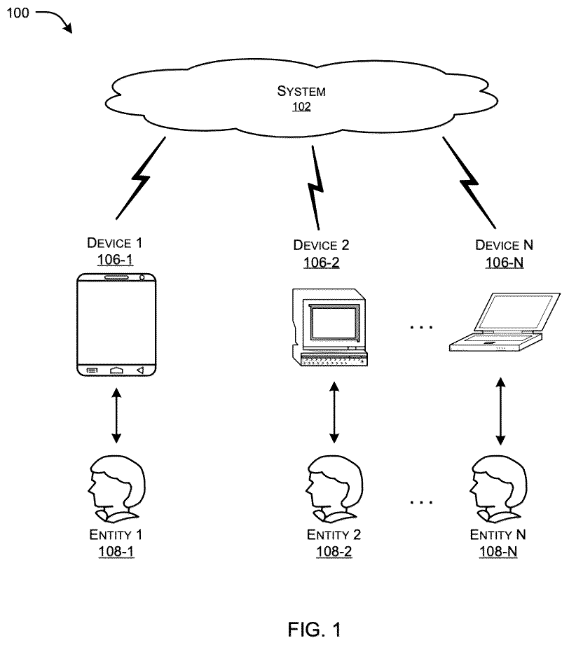

FIG. 1 illustrates a network implementation 100 of a system which facilitates to provide guidance regarding cardiovascular disease prevention, in accordance with an embodiment of the present disclosure.

According to a network implementation 100, a system 102 can facilitate guidance regarding cardiovascular disease prevention, based on national and international medical guidelines. Although the present subject matter is explained considering that the system 102 is implemented as an application on a server, it may be understood that the system 102 may also be implemented in a variety of computing systems, such as a laptop computer, a desktop computer, a notebook, a workstation, a server, a network server, a cloud-based environment and the likes. The system 102 being implemented through a website on browser like Google Chrome, Safari and the likes in a variety of devices such as mobile phone, tablet, computer laptop and the likes. It would be appreciated that the system 102 may be accessed by multiple users (also referred to as entities) 108-1, 108-2 . . . 108-N (collectively referred to as users 108, and individually referred to as the user 108 hereinafter), through one or more computing devices 106-1, 106-2 . . . 106-N (collectively referred to as computing devices 106 and individually referred to as computing device 106, hereinafter), or applications residing on the computing devices 106. Examples of the computing devices 106 may include, but are not limited to, a portable computer, a personal digital assistant, a mobile terminal, a handheld device, and a workstation. The computing device 106 can be communicatively coupled with the system 102 through a network 104.

In one implementation, the network 104 can be a wireless network, a wired network or a combination thereof. The network 104 can be implemented as one of the different types of networks, such as intranet, local area network (LAN), wide area network (WAN), the internet, and the like. Further, the network 104 may either be a dedicated network or a shared network. The shared network represents an association of the different types of networks that use a variety of protocols, for example, Hypertext Transfer Protocol (HTTP), Transmission Control Protocol/Internet Protocol (TCP/IP), Wireless Application Protocol (WAP), and the like, to communicate with one another. Further the network 104 can include a variety of network devices, including routers, bridges, servers, computing devices, storage devices, and the like. In another implementation the network 104 can be cellular network or mobile communication network based on various technologies, including but not limited to, Global System for Mobile (GSM), General Packet Radio Service (GPRS), Code Division Multiple Access (CDMA), Long Term Evolution (LTE), WiMAX, and the like.

In an implementation, the user 108 can access the system 102 through set of instructions residing on the computing device 106. The user 108 can self-register himself with the system 102 using identity attributes, where the identity attributes include name, age, gender, mobile number, address and other related information.

User 108 can enter the following set of attributes/information to the set of instructions residing on the computing device 106. The set of attributes can include medical history information, blood test information, imaging test information, current medications and any combination thereof. The medical history information can include demographics like age, gender, ethnicity, medical history of heart disease including procedures/surgery, other relevant medical histories, family history, assessment of lifestyle risk factors like smoking, exercise, alcohol, blood pressure measurement, height, weight and any combination thereof.

The blood test information can include lipid profile, extended lipid profile, diabetes blood tests like blood sugar, haemoglobin A1C, liver function tests, blood chemistry like electrolytes and creatinine and any combination thereof.

The imaging test information can include carotid ultrasound and computed tomography (CT) calcium score.

The current medications can include statins, other cholesterol/lipid/triglyceride medication (such as, Ezetimibe, Fibrate), blood pressure medications, diabetes medications and the likes.

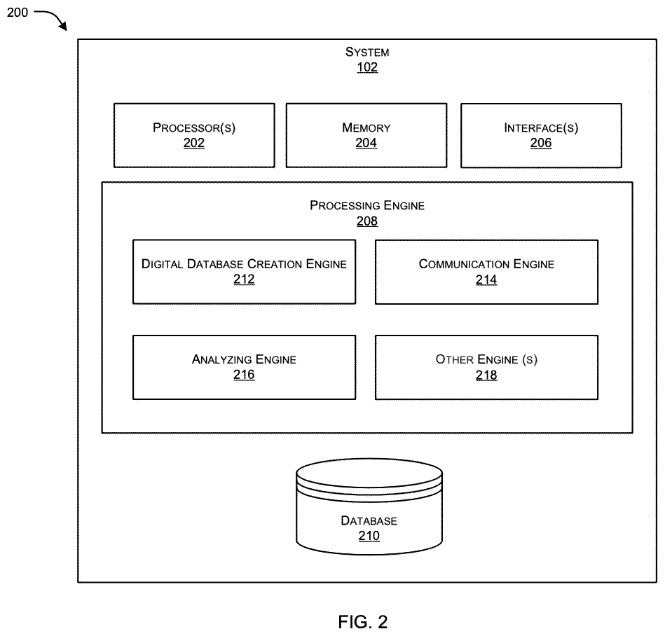

In an embodiment, the system 102 implemented on the computing device 106 that can include a processor 202 operatively coupled to a memory 204 (as illustrated in FIG. 2), the memory 204 storing instructions executable by the processor 202 to receive the set of attributes from the user 108 associated with the computing device 106. The set of attributes can include medical history information, blood test information, imaging test information, current medication and any combination thereof.

The processor 202 can receive the set of attributes from the user and analyse the received set of attributes to stratify the user into one or more risk categories based on the analyzed set of attributes and the calculated risk percent of cardiovascular disease, where based on the stratification of the user into one or more risk categories, the processor 202 is configured to assess medications for the user to help achieve lipid control targets recommended by the medical guidelines.

The risk percent of cardiovascular disease is calculated using the appropriate risk scoring system available. The processor 202 is configured to guide the user regarding the appropriate intensity of medications and the recommended dose, where the medications are statin and any combination thereof. The processor 202 recommends changes to the medication according to targets for lipid control set by evidence-based national and international medical guidelines. The processor 202 monitors adverse effects of the medications to ensure safety, wherein when the adverse effects are detected, appropriate action is recommended. The processor 202 assesses risk factors for elevated triglycerides and appropriate lifestyle and medications are recommended. Further, processor 202 is configured to provide recommendation to user 108 to consult an expert specialist, whenever the clinical scenario depends on the clinical judgement of the specialist. The system 102 can provide service to doctors and other users via telehealth means.

The embodiments of the present disclosure described above provide several advantages. The one or more of the embodiments provides the system 102 that enables the user with elevated risk and early-stage disease to receive timely screening and timely prevention of cardiovascular disease. The system 102 equips all doctors to provide gold-standard cardiovascular disease prevention advice to all patients. The system 102 reduces the enormous burden that falls on the limited number of cardiologists and the system serves as an educational tool for non-cardiac doctors and common people to learn more about heart disease risk, risk factors, means to reduce risk and control risk factors.

FIG. 2 illustrates exemplary functional components 200 of the proposed system in accordance with an embodiment of the present disclosure.

In an aspect, the system 102 may comprise one or more processor(s) 202. The one or more processor(s) 202 may be implemented as one or more microprocessors, microcomputers, microcontrollers, digital signal processors, central processing units, logic circuitries, and/or any devices that manipulate data based on operational instructions. Among other capabilities, the one or more processor(s) 202 are configured to fetch and execute computer-readable instructions stored in a memory 204 of the system 102. The memory 204 may store one or more computer-readable instructions or routines, which may be fetched and executed to create or share the data units over a network service. The memory 204 may comprise any non-transitory storage device including, for example, volatile memory such as RAM, or non-volatile memory such as EPROM, flash memory, and the like.

The system 102 may also comprise an interface(s) 206. The interface(s) 206 may comprise a variety of interfaces, for example, interfaces for data input and output devices, referred to as I/O devices, storage devices, and the like. The interface(s) 206 may facilitate communication of system 102. The interface(s) 206 may also provide a communication pathway for one or more components of the system 102. Examples of such components include, but are not limited to, processing engine(s) 208 and data 210.

The processing engine(s) 208 may be implemented as a combination of hardware and programming (for example, programmable instructions) to implement one or more functionalities of the processing engine(s) 208. In examples described herein, such combinations of hardware and programming may be implemented in several different ways. For example, the programming for the processing engine(s) 208 may be processor executable instructions stored on a non-transitory machine-readable storage medium and the hardware for the processing engine(s) 208 may comprise a processing resource (for example, one or more processors), to execute such instructions. In the present examples, the machine-readable storage medium may store instructions that, when executed by the processing resource, implement the processing engine(s) 208. In such examples, the system 102 may comprise the machine-readable storage medium storing the instructions and the processing resource to execute the instructions, or the machine-readable storage medium may be separate but accessible to system 102 and the processing resource. In other examples, the processing engine(s) 208 may be implemented by electronic circuitry.

The data 210 may comprise data that is either stored or generated as a result of functionalities implemented by any of the components of the processing engine(s) 208 or the system 102. In an exemplary embodiment, the processing engine(s) 208 may include a database creation engine 212, a communication engine 214, analysing engine 216 and other engine(s) 218.

In an embodiment, the database creation engine 212 facilitates to create a database related to information of users; a communication engine 214 facilitates the users to provide their information. The analysing engine 216 analyses the information of the user and maintains a record of the same for future reference and use. The other engine(s) 218 can supplement the functionalities of the processing engine 208 or the system 102.

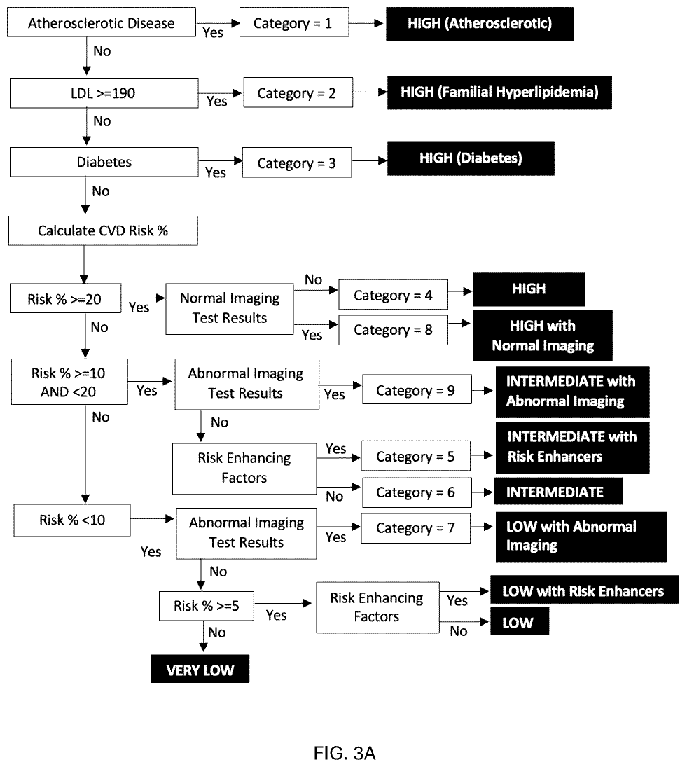

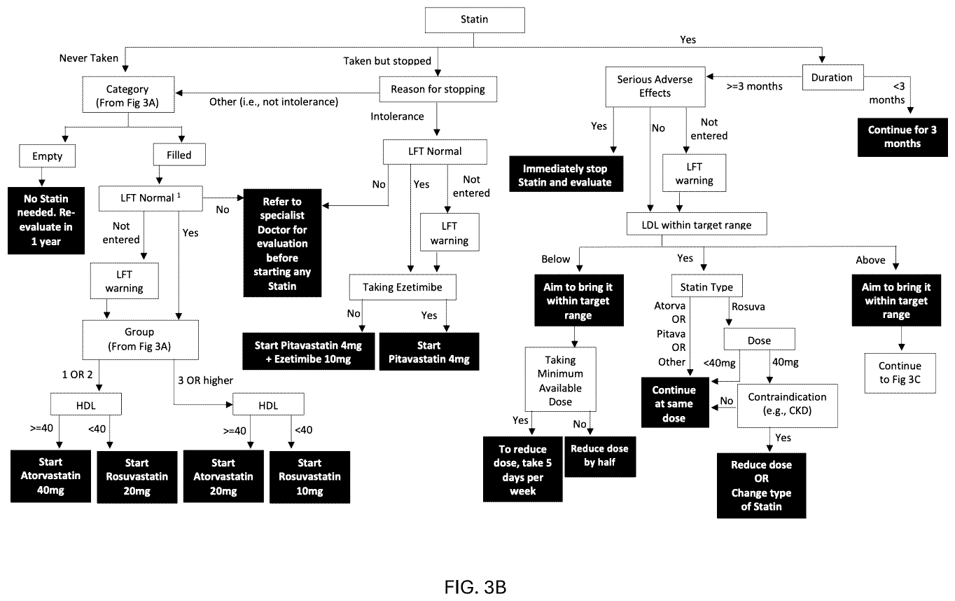

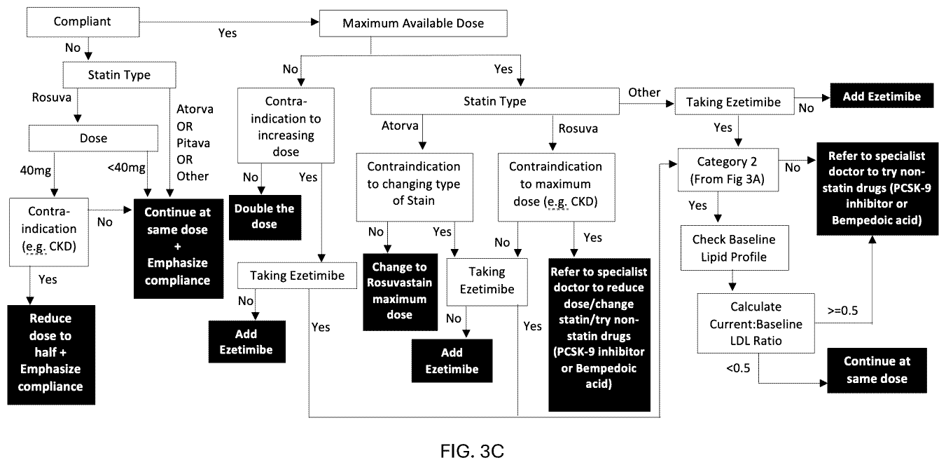

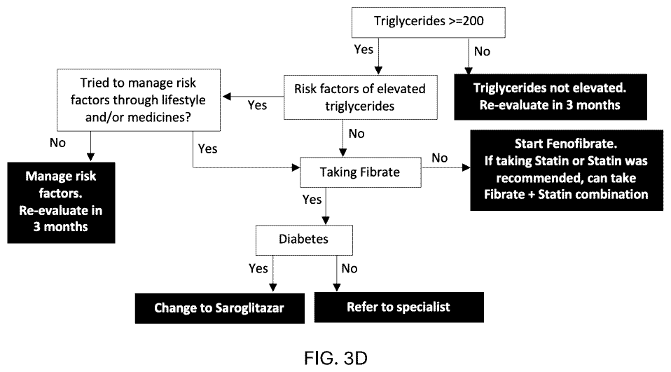

FIG. 3A illustrates a framework 300 for stratification of user into risk categories, in accordance with an embodiment of the present disclosure.

The processor 202 based on the above information provided by the user can determine whether the user/patient needs to be assessed for statin indication i.e., whether he/she would benefit from starting statin for prevention or the user/patient needs to be assessed for adequate control with a statin, if the user is already taking statin. If the user needs to be assessed for statin indication, based on all of the above information provided cardiovascular disease risk is calculated and the user is stratified into risk categories.

Cardiovascular disease risk percent: the risk of developing cardiovascular disease is calculated using a published and validated risk scoring system.

Risk categories: Based on the medical history and calculated cardiovascular disease risk percent, the user can be stratification into groups or risk categories/level as shown in FIG. 3A. The one or more groups include atherosclerotic disease that refers to any cardiovascular disease involving atherosclerosis of the arteries.

Diabetes: Based on reported history of diabetes mellitus and/or blood test results meeting criteria for diagnosis.

Normal imaging test results: Imaging test results are classified as normal if maximum stenosis on carotid ultrasound=0 and/or CT calcium score=0 and/or imaging test(s) has not been done.

Abnormal imaging test results: Imaging test results are classified as abnormal if maximum stenosis on carotid ultrasound >0 and/or CT calcium score >0 and/or patient or user reports history of abnormal result.

Further, the risk enhancing factors considered in the stratification process include, but are not limited to:
strong family history of early heart disease
high low-density lipoprotein (LDL)
low high-density lipoprotein (HDL)
elevated biomarkers [such as Apolipoprotein B, Lipoprotein (A), high sensitivity-C Reactive Protein]
metabolic syndrome
atrial fibrillation
migraine
history of preeclampsia
early menopause (before 40 years of age)
chronic inflammatory diseases
prolonged use of corticosteroids.

The user is stratified into any one of the groups of risk level based on the medical history and the calculated percent of the cardiovascular disease.

FIG. 3B illustrates a framework 302 for providing guidance regarding the appropriate type/intensity of statin and the recommended dose, in accordance with an embodiment of the present disclosure. Based on the risk stratification, the user is given guidance regarding the appropriate type/intensity of statin and the recommended dose to follow as shown in FIG. 3B. If the user indicates that the patient is already taking statin, control with statins is assessed according to targets set by evidence-based National and International Medical Guidelines. Accordingly, a change in dose or change of the type of statin is recommended as needed to achieve said targets. Additionally, monitoring for adverse effects of statins is carried out to ensure safety. If adverse effects are detected, appropriate action is recommended. Furthermore, whenever the clinical scenario depends on the clinical judgement of a specialist cardiologist, the user is advised to consult an expert cardiologist.

Serious adverse effects are assessed on the basis of symptoms reported by the user (such as, severe muscle pain and cramps or myoglobinuria) and/or liver enzymes blood test results (SGOT and SGPT) reported by the user.

Intolerance refers to statin intolerance i.e., patient experiencing any of the serious adverse effects when statin was taken in the past.

Chronic kidney disease (CKD) includes patient reported history of chronic kidney disease or creatinine above threshold for CKD.

Compliance refers to patient taking prescribed medication (statin) regularly and maintaining heart healthy lifestyle (regular exercise and healthy diet).

Other refers to brand name of statin being taken by patient, which is not in the list provided by the website/application.

FIG. 3C illustrates a framework 304 for elevated triglycerides, in accordance with an embodiment of the present disclosure. Referring to FIG. 3C for elevated triglycerides, risk factors are assessed and appropriate lifestyle and/or medications are recommended as shown below in flowchart 3C. The risk factors for elevated triglycerides include sedentary lifestyle i.e., lack of regular exercise, excess alcohol intake, hypothyroidism i.e., low thyroid hormone, diabetes, overweight/obesity.

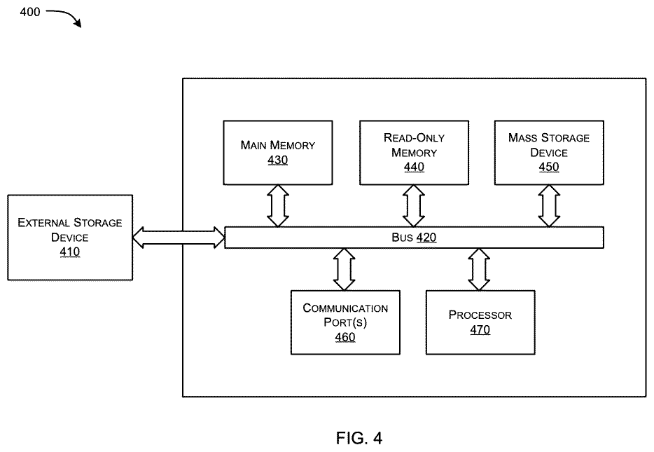

FIG. 4 illustrates an exemplary computer system 400 to implement the proposed system in accordance with embodiments of the present disclosure.

As shown in FIG. 4, computer system can include an external storage device 410, a bus 420, a main memory 430, a read only memory 440, a mass storage device 450, communication port 460, and a processor 470. A person skilled in the art will appreciate that computer system may include more than one processor and communication ports. Examples of processor 470 include, but are not limited to, an Intel® Itanium® or Itanium 2 processor(s), or AMD® Opteron® or Athlon MP® processor(s), Motorola® lines of processors, FortiSOC™ system on a chip processors or other future processors. Processor 470 may include various modules associated with embodiments of the present invention. Communication port 460 can be any of an RS-232 port for use with a modem-based dialup connection, a 10/100 Ethernet port, a Gigabit or 10 Gigabit port using copper or fibre, a serial port, a parallel port, or other existing or future ports. Communication port 460 may be chosen depending on a network, such a Local Area Network (LAN), Wide Area Network (WAN), or any network to which computer system connects.

Memory 430 can be Random Access Memory (RAM), or any other dynamic storage device commonly known in the art. Read only memory 440 can be any static storage device(s) e.g., but not limited to, a Programmable Read Only Memory (PROM) chips for storing static information e.g., start-up or BIOS instructions for processor 470. Mass storage 450 may be any current or future mass storage solution, which can be used to store information and/or instructions. Exemplary mass storage solutions include, but are not limited to, Parallel Advanced Technology Attachment (PATA) or Serial Advanced Technology Attachment (SATA) hard disk drives or solid-state drives (internal or external, e.g., having Universal Serial Bus (USB) and/or Firewire interfaces), e.g. those available from Seagate (e.g., the Seagate Barracuda 7102 family) or Hitachi (e.g., the Hitachi Deskstar 7K1000), one or more optical discs, Redundant Array of Independent Disks (RAID) storage, e.g. an array of disks (e.g., SATA arrays), available from various vendors including Dot Hill Systems Corp., LaCie, Nexsan Technologies, Inc. and Enhance Technology, Inc.

Bus 420 communicatively couples processor(s) 470 with the other memory, storage and communication blocks. Bus 420 can be, e.g. a Peripheral Component Interconnect (PCI)/PCI Extended (PCI-X) bus, Small Computer System Interface (SCSI), USB or the like, for connecting expansion cards, drives and other subsystems as well as other buses, such a front side bus (FSB), which connects processor 470 to software system.

Optionally, operator and administrative interfaces, e.g. a display, keyboard, and a cursor control device, may also be coupled to bus 420 to support direct operator interaction with computer system. Other operator and administrative interfaces can be provided through network connections connected through communication port 460. External storage device 410 can be any kind of external hard-drives, floppy drives, IOMEGA® Zip Drives, Compact Disc-Read Only Memory (CD-ROM), Compact Disc-Re-Writable (CD-RW), Digital Video Disk-Read Only Memory (DVD-ROM). Components described above are meant only to exemplify various possibilities. In no way should the aforementioned exemplary computer system limit the scope of the present disclosure.

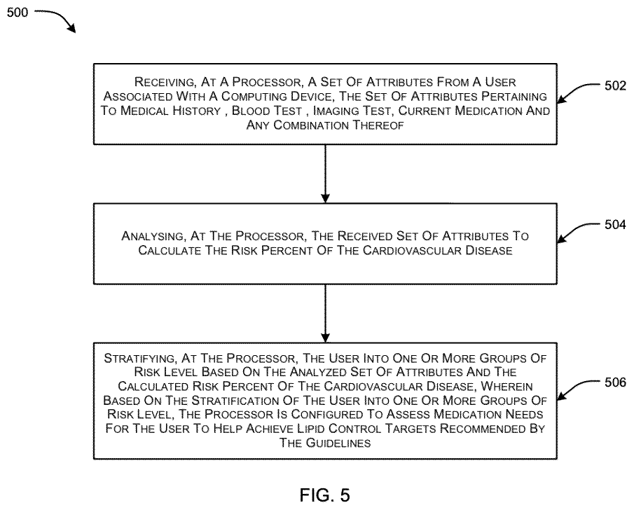

FIG. 5 illustrates a flow chart of method for providing guidance regarding cardiovascular disease prevention, in accordance with an embodiment of the present disclosure.

Referring to FIG. 5, at block 502, the processor can receive the set of attributes from the user associated with the computing device, the set of attributes pertaining to medical history information, blood test information, imaging test information, current medication and any combination thereof.

At block 504, the processor 504 can analyse the received set of attributes to calculate the risk percent of developing cardiovascular disease. At block 506, the processor can stratify the user into one or more risk categories based on the analyzed set of attributes and the calculated risk percent of the cardiovascular disease, where based on the stratification of the user into one or more groups of risk level, the processor is configured to assess medication needs for the user to help achieve lipid control targets recommended by the medical guidelines.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### Advantages of the Present Disclosure

The present disclosure provides a system that enables the user with elevated risk and early-stage disease to receive timely screening and timely prevention of cardiovascular disease.

The present disclosure provides a system that includes an evaluation of risk-enhancing factors in addition to traditional risk factors

The present disclosure provides a system that enables the assessment of the medication needs of the patient to attain guideline recommended lipid targets to effectively reduce cardiovascular disease risk

The present disclosure provides a system that removes the knowledge variability and equips all doctors to provide gold-standard cardiovascular disease prevention advice to all patients.

The present disclosure provides a system that can ensure that all patients receive cardiologist-level of prevention even if they are being seen by primary care physicians or other non-physician providers (such as, Nurse Practitioners or Physician Assistants).

The present disclosure provides a system that reduces the enormous burden that falls on the limited number of cardiologists.

The present disclosure provides a system that serves as an educational tool for non-cardiac doctors and common people to learn more about heart disease risk, risk factors, means to reduce risk and control risk factors.

We claim:

1. A system (102) for providing guidance of cardiovascular disease prevention, said system comprising:
a processor (202) operatively coupled to a memory (204), the memory storing instructions executable by the processor (202) to:
receive a set of attributes from a user (108) associated with a computing device (106), the set of attributes pertaining to medical history information, blood test information, imaging test information, current medication and any combination thereof;
analyse the received set of attributes to stratify the user (108) into one or more risk categories based on the analyzed set of attributes and the calculated risk percent of the cardiovascular disease, wherein based on the stratification of the user into one or more risk categories, the processor is configured to:
assess medication needs for the user to help achieve lipid control targets recommended by medical guidelines;
guide the user regarding the intensity of medications and the recommended dose; and
recommend changes to the intensity of medications and the recommended dose according to targets for lipid control set.

2. The system as claimed in claim 1, wherein the processor (202) monitors adverse effects of the medications to ensure safety, wherein when the adverse effects are detected, appropriate action is recommended.

3. The system as claimed in claim 1, wherein the processor (202) assesses risk factors for elevated triglycerides and appropriate lifestyle and medications are recommended.

4. The system as claimed in claim 1, wherein the processor (202) configured to recommend the user to consult an expert specialist, whenever the clinical scenario depends on the clinical judgement of the specialist.

5. A method (500) for providing guidance of cardiovascular disease prevention, the method comprising:
receiving (502), at a processor, a set of attributes from a user associated with a computing device, the set of attributes pertaining to medical history information, blood test information, imaging test information, current medication and any combination thereof;
analysing (504), at the processor, the received set of attributes to stratify the user into one or more risk categories based on the analyzed set of attributes and the calculated risk percent of the cardiovascular disease, wherein based on the stratification of the user into one or more groups of risk level, the processor is configured to assess medication needs for the user to help achieve lipid control targets recommended by medical guidelines.
